(19) United States
(12) Patent Application Publication
(10) Pub. No.: US 2024/0242341 A1
(43) Pub. Date: Jul. 18, 2024

(54) IMAGE ANALYSIS SUPPORT APPARATUS, IMAGE ANALYSIS SUPPORT SYSTEM, AND IMAGE ANALYSIS SUPPORT METHOD

(71) Applicant: Canon Medical Systems Corporation, Otawara-shi (JP)

(72) Inventors: Masashi NOBE, Utsunomiya (JP); Yasunari KUTSUNA, Nasushiobara (JP); Junichi TASHIRO, Otawara (JP); Yosuke YANAGIDA, Nasushiobara (JP); Tomomi HASHIMOTO, Nasushiobara (JP)

(73) Assignee: Canon Medical Systems Corporation, Otawara-shi (JP)

(21) Appl. No.: 18/415,113

(22) Filed: Jan. 17, 2024

(30) Foreign Application Priority Data
Jan. 17, 2023 (JP) 2023-005236

(51) Int. Cl. G06T 7/00; G06T 7/11; G06T 7/73

(52) U.S. Cl. CPC G06T 7/0012; G06T 7/11; G06T 7/73; G06T 2207/30096

(57) ABSTRACT

According to one embodiment, an image analysis support apparatus includes processing circuitry. The processing circuitry acquires a first analysis result obtained through analysis of medical image data performed by a first analysis application. Based on the acquired first analysis result, the processing circuitry determines at least one candidate relating to a second analysis application that further analyzes the medical image data.

### CROSS-REFERENCE TO RELATED APPLICATION(S)

This application is based upon and claims the benefit of priority from Japanese Patent Application No. 2023-005236, filed Jan. 17, 2023, the entire contents of which are incorporated herein by reference.

### FIELD

Embodiments described herein relate generally to an image analysis support apparatus, an image analysis support system, and an image analysis support method.

### BACKGROUND

In general, various analysis applications (analysis programs) for analyzing medical image data have been introduced into medical facilities. An operator in a medical facility may wish to analyze medical image data by combining multiple analysis applications. For example, an operator may wish to compute feature information of a lesion region using an analysis application for additional analysis after extracting the lesion region from medical image data using an analysis application for initial analysis.

Conventionally, an analysis application for additional analysis has been automatically selected according to the scan information (e.g., imaging conditions, examination information) of medical image data. In this method, however, the selected analysis application for additional analysis may not match an operator's preference. For example, if medical image data has scan information relating to “brain tumor”, an analysis application for “brain tumor” is selected. However, the operator may wish to use an analysis application for “brain aneurysm”.

### BRIEF DESCRIPTION OF THE DRAWINGS

FIG. 1 is a block diagram showing an example of a configuration of an image analysis support system according to an embodiment.

FIG. 2 is a block diagram showing an example of a configuration of an image analysis support apparatus according to an embodiment.

FIG. 3 is a block diagram showing an example of a configuration of an operation terminal according to an embodiment.

FIG. 4 is a sequence diagram showing a first operation example of the image analysis support system according to an embodiment.

FIG. 5 is a diagram showing a first display example of a candidate list according to an embodiment.

FIG. 6 is a diagram showing a second display example of a candidate list according to an embodiment.

FIG. 7 is a diagram showing a third display example of a candidate list according to an embodiment.

FIG. 8 is a sequence diagram showing a second operation example of the image analysis support system according to an embodiment.

### DETAILED DESCRIPTION

In general, according to one embodiment, an image analysis support apparatus includes processing circuitry. The processing circuitry acquires a first analysis result obtained through analysis of medical image data performed by a first analysis application. Based on the acquired first analysis result, the processing circuitry determines at least one candidate relating to a second analysis application that further analyzes the medical image data.

Hereinafter, an image analysis support apparatus, an image analysis support system, and an image analysis support method according to an embodiment will be described with reference to the accompanying drawings. In the embodiment(s) described below, elements assigned the same reference numerals perform the same operations, and redundant descriptions will be omitted as appropriate.

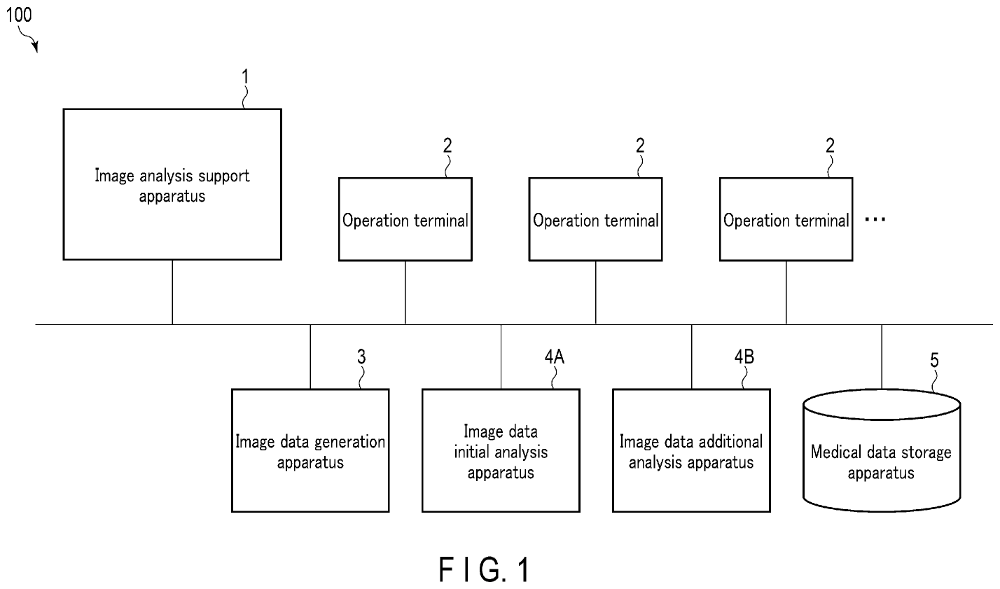

FIG. 1 is a block diagram showing an example of a configuration of an image analysis support system 100 according to an embodiment. The image analysis support system 100 is a system that supports image analysis performed by multiple analysis applications. The image analysis support system 100 is a client server system. The image analysis support system 100 includes, as its components, an image analysis support apparatus 1, multiple operation terminals 2, an image data generation apparatus 3, an image data initial analysis apparatus 4A, an image data additional analysis apparatus 4B, and a medical data storage apparatus 5. These components are connected to one another via a bus, which is a common signal transmission path, in such a manner as to be able to communicate with one another.

The image analysis support apparatus 1 is an apparatus that supports image analysis performed by multiple analysis applications. The image analysis support apparatus 1 functions as a server in the image analysis support system 100. The image analysis support apparatus 1 may be a workstation capable of performing high-speed information processing. The image analysis support apparatus 1 is also referred to as “a medical information processing apparatus”.

The operation terminal 2 is a terminal operated by an operator (e.g., a doctor, a technician). The operation terminal 2 functions as a client in the image analysis support system 100. The operation terminal 2 may be a desktop PC, a laptop PC, a smartphone, a tablet terminal, or a wearable terminal.

The image data generation apparatus 3 is an apparatus that generates medical image data (hereinafter abbreviated as “image data”). The image data generation apparatus 3 may be a medical diagnostic imaging apparatus (e.g., an X-ray diagnostic apparatus, an X-ray CT apparatus, an MRI apparatus, an ultrasonic diagnostic apparatus, a nuclear medicine examination apparatus). The image data generation apparatus 3 transmits the generated image data to the medical data storage apparatus 5.

The image data initial analysis apparatus 4A is an apparatus that initially analyzes the image data. The image data initial analysis apparatus 4A includes one initial-analysis application A1. The initial-analysis application A1 extracts, as an analysis result, a position of an organ region (e.g., brain, heart, lung), a lesion region (e.g., hematocele, tumor), or an anatomical landmark from the image data. The extracted position may be represented as positional information on a patient's coordinate system. A known image recognition technique (e.g., image classification, object detection, segmentation) may be applied to this extraction. This analysis result may include the name or type of the initial-analysis application A1. The image data initial analysis apparatus 4A transmits the analysis result provided by the initial-analysis application A1 (“initial-analysis result R1”) to the medical data storage apparatus 5.

The image data additional analysis apparatus 4B is an apparatus that additionally analyzes the image data using the analysis result provided by the initial-analysis application A1. The image data additional analysis apparatus 4B includes multiple additional-analysis applications A2. Each of the additional-analysis application A2 performs various physiological analyses (e.g., bloodstream analysis, respiration analysis) or morphological analysis (e.g., three-dimensional rendering) on the organ region, lesion region, or anatomical landmark extracted by the initial-analysis application A1. The image data additional analysis apparatus 4B transmits the analysis result provided by the additional-analysis application A2 (“additional-analysis result R2”) to the medical data storage apparatus 5.

The medical data storage apparatus 5 is an apparatus that stores the medical data (e.g. image data, analysis result). The medical data storage apparatus 5 may be a storage medium (e.g., a magnetic storage medium, an electromagnetic storage medium, an optical storage medium, a semiconductor memory) or a drive that reads and writes information to and from a storage medium. The medical data storage apparatus 5 stores the image data transmitted from the image data generation apparatus 3 and the analysis result transmitted from the image data initial analysis apparatus 4A or the image data additional analysis apparatus 4B.

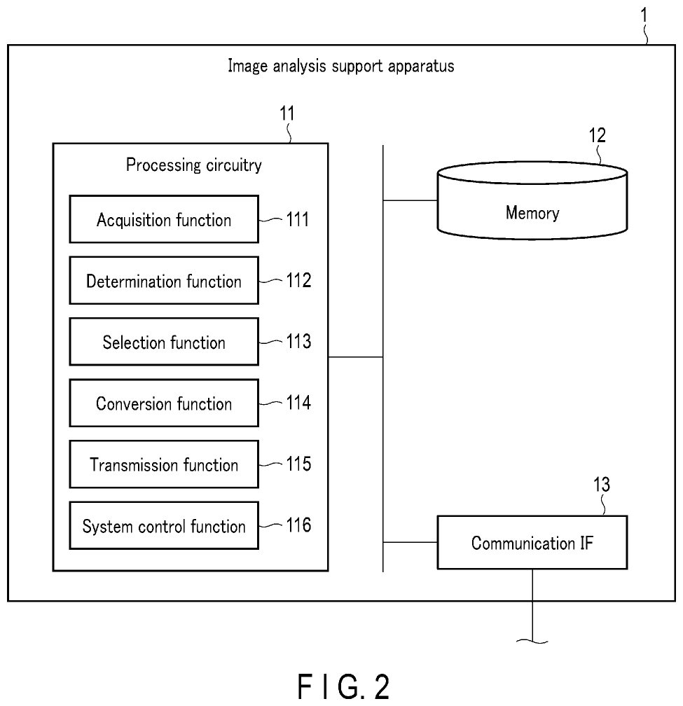

FIG. 2 is a block diagram showing an example of a configuration of the image analysis support apparatus 1 according to an embodiment. The image analysis support apparatus 1 includes processing circuitry 11, a memory 12, and a communication IF 13 as its components. These components are connected to one another via a bus, which is a common signal transmission path, in such a manner as to be able to communicate with one another.

The processing circuitry 11 is circuitry that controls the entire operation of the image analysis support apparatus 1. The processing circuitry 11 includes at least one processor. The processor refers to circuitry such as a CPU (central processing unit), a GPU (graphics processing unit), an ASIC (application specific integrated circuit), or a programmable logic device (for example, an SPLD (simple programmable logic device), a CPLD (complex programmable logic device), or an FPGA (field programmable gate array)). If the processor is a CPU, the CPU reads and executes the programs stored in the memory 12 to implement the functions. If the processor is an ASIC, each function is directly incorporated as logic circuitry into the circuitry of the ASIC. The processor may be constituted in the form of single circuitry or in the form of multiple independent sets of circuitry combined. The processing circuitry 11 implements each function (an acquisition function 111, a determination function 112, a selection function 113, a conversion function 114, a transmission function 115, and a system control function 116).

The acquisition function 111 is a function of acquiring various kinds of data or information. For example, the acquisition function 111 acquires the initial-analysis result R1 obtained by the analysis of image data performed by the initial-analysis application A1. The acquisition function 111 may acquire various kinds of data or information by accessing the medical data storage apparatus 5. The acquisition function 111 is an example of an acquisition unit.

The determination function 112 is a function of determining various contents. For example, the determination function 112 determines, based on the initial-analysis result R1 acquired by the acquisition function 111, at least one candidate relating to the additional-analysis application A2 that further analyzes the image data. The determination function 112 is an example of a determination unit.

The selection function 113 is a function of selecting various kinds of data or information. For example, the selection function 113 selects, among the at least one candidate determined by the determination function 112, one candidate according to a selection operation performed by an operator. The selection function 113 is an example of a selection unit.

The conversion function 114 is a function of converting various kinds of data or information. For example, the conversion function 114 converts the data format of the initial-analysis result R1 such that the data format of the initial-analysis result R1 matches the data format that can be understood by the additional-analysis application A2 corresponding to the candidate selected by the selection function 113. The conversion function 114 is an example of a conversion unit.

The transmission function 115 is a function of transmitting various kinds of data or information. For example, the transmission function 115 transmits the initial-analysis result R1 converted by the conversion function 114 to the additional-analysis application A2 corresponding to the candidate selected by the selection function 113. The transmission function 115 is an example of a transmission unit.

The system control function 116 is a function of controlling various operations performed by the processing circuitry 11. For example, the system control function 116 provides an operating system (OS) for the processing circuitry 11 to implement each function (the acquisition function 111, the determination function 112, the selection function 113, the conversion function 114, and the transmission function 115). The system control function 116 is an example of a system controller.

The memory 12 is a device for storing various kinds of data or information. The memory 12 may be a processor-readable storage medium (e.g., a magnetic storage medium, an electromagnetic storage medium, an optical storage medium, a semiconductor memory) or a drive that reads and writes data or information to and from a storage medium. The memory 12 stores each program for causing the processing circuitry 11 to implement each function (the acquisition function 111, the determination function 112, the selection function 113, the conversion function 114, the transmission function 115, and the system control function 116). The memory 12 is an example of a storage unit.

The communication IF 13 is an interface that communicates various kinds of data or information with each of the components included in the image analysis support system 100. The communication IF 13 communicates various kinds of data or information with the operation terminals 2 or the medical data storage apparatus 5. The communication IF 13 is an example of a communication unit.

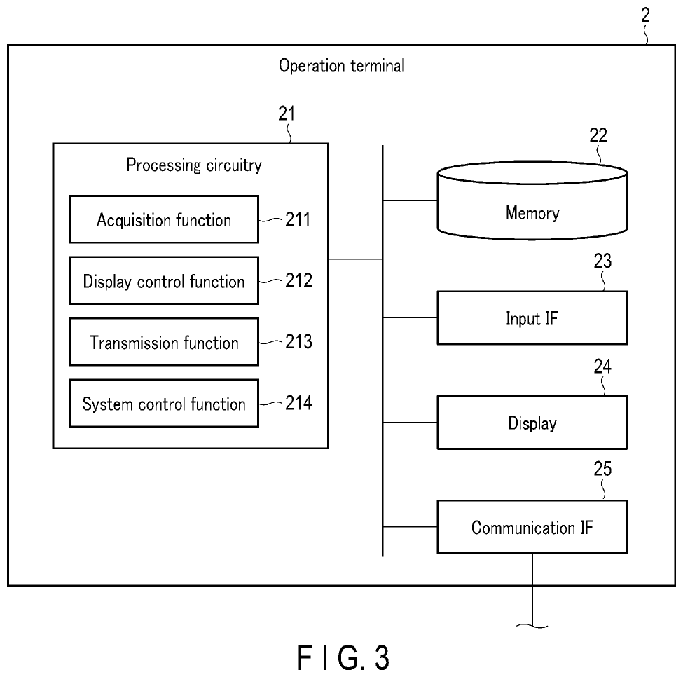

FIG. 3 is a block diagram showing an example of a configuration of the operation terminal 2 according to an embodiment. The operation terminal 2 includes processing circuitry 21, a memory 22, an input IF 23, a display 24, and a communication IF 25 as its components. These components are connected to one another via a bus, which is a common signal transmission path, in such a manner as to be able to communicate with one another.

The processing circuitry 21 is circuitry that controls the entire operation of the operation terminal 2. The processing circuitry 21 of the operation terminal 2 has a hardware configuration similar to that of the processing circuitry 11 of the image analysis support apparatus 1. The processing circuitry 21 implements each function (an acquisition function 211, a display control function 212, a transmission function 213, and a system control function 214).

The acquisition function 211 is a function of acquiring various kinds of data or information. For example, the acquisition function 211 acquires at least one candidate relating to the additional-analysis application A2 from the image analysis support apparatus 1. The acquisition function 211 may acquire display data for listing these candidates from the image analysis support apparatus 1. The acquisition function 211 is an example of an acquisition unit.

The display control function 212 is a function of causing various kinds of data or information to be displayed. For example, the display control function 212 causes the at least one candidate acquired by the acquisition function 211 to be displayed. The display control function 212 may cause the display 24 to display a display image based on the display data for listing these candidates. The display control function 212 is an example of a display controller.

The transmission function 213 is a function of transmitting various kinds of data or information. For example, the transmission function 213 transmits a selection operation performed by an operator to the image analysis support apparatus 1. The transmission function 213 is an example of a transmission unit.

The system control function 214 is a function of controlling various operations performed by the processing circuitry 21. For example, the system control function 214 provides an operating system (OS) for the processing circuitry 21 to implement each function (the acquisition function 211, the display control function 212, and the transmission function 213). The system control function 214 is an example of a system controller.

The memory 22 is a device for storing various kinds of data and information. The memory 22 of the operation terminal 2 has a hardware configuration similar to that of the memory 12 of the image analysis support apparatus 1. The memory 22 stores each program for causing the processing circuitry 21 to implement each function (the acquisition function 211, the display control function 212, the transmission function 213, and the system control function 214). The memory 22 is an example of a storage unit.

The input IF 23 is an interface for receiving various operations from an operator. The input IF 23 converts various operations received from an operator into electric signals, and transmits the electric signals to the processing circuitry 21. The input IF 23 may be a mouse, a keyboard, a button, a panel switch, a slider switch, a trackball, an operation panel, or a touch panel. The input IF 23 is an example of an input unit.

The display 24 is a device for displaying various kinds of data or information. The display 24 may be a liquid crystal display, a plasma display, an organic EL display, or an LED display. The display 24 may be a touch-panel display that also serves as the input IF 23. The display 24 is an example of a display unit.

The communication IF 25 is an interface that communicates various kinds of data or information with each of the components included in the image analysis support system 100. The communication IF 25 communicates various kinds of data or information with the image analysis support apparatus 1. The communication IF 25 is an example 10 of a communication unit.

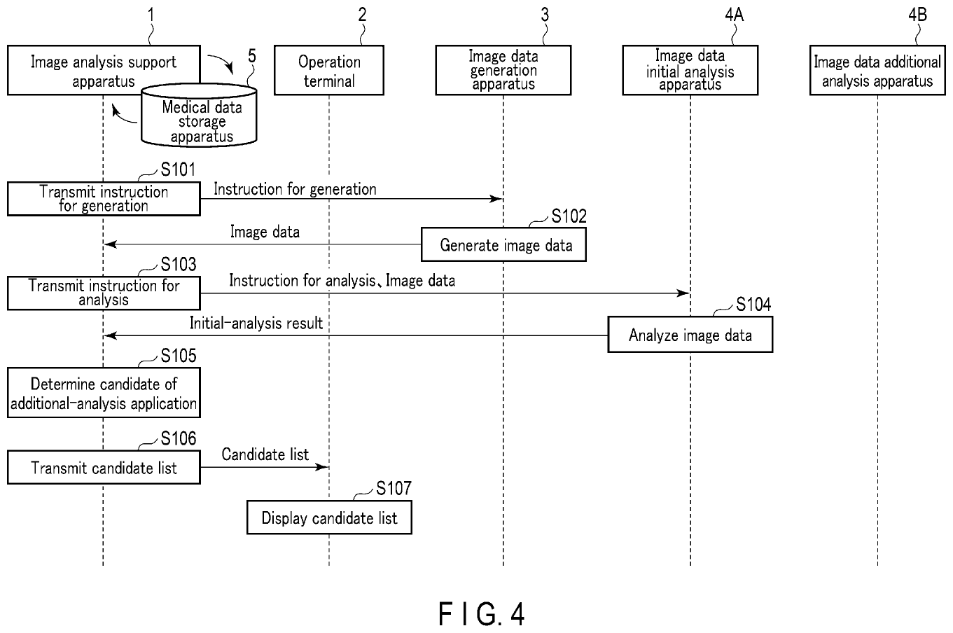

FIG. 4 is a sequence diagram showing a first operation example of the image analysis support system 100 according to the embodiment. In this operation example, the image analysis support apparatus 1 communicates various kinds of data or information with the medical data storage apparatus 5. This operation may be started when the image analysis support apparatus 1 acquires, from the operation terminal 2, a request indicating that image data analysis is desired.

(Step S101) First, the image analysis support apparatus 1 transmits an instruction for generation. Specifically, by implementing the transmission function 115, the image analysis support apparatus 1 transmits an instruction for generating image data to the image data generation apparatus 3.

(Step S102) Next, the image data generation apparatus 3 generates image data. Specifically, the image data generation apparatus 3 generates image data according to the instruction for generation transmitted in step S101. The image data generation apparatus 3 transmits the generated image data to the medical data storage apparatus 5. The transmitted image data is acquired by the acquisition function 111 of the image analysis support apparatus 1.

(Step S103) Subsequently, the image analysis support apparatus 1 transmits an instruction for analysis. Specifically, by implementing the transmission function 115, the image analysis support apparatus 1 transmits an instruction for analyzing the image data generated in step S102 to the image data initial analysis apparatus 4A. Simultaneously, by implementing the transmission function 115, the image analysis support apparatus 1 transmits the image data generated in step 5102 to the image data initial analysis apparatus 4A.

(Step S104) Subsequently, the image data initial analysis apparatus 4A analyzes the image data. Specifically, the image data initial analysis apparatus 4A initially analyzes the image data transmitted in step S103 according to the instruction for analysis transmitted in step 5103. The image data initial analysis apparatus 4A transmits the initial-analysis result R1 of the image data provided by the initial-analysis application A1 to the medical data storage apparatus 5. The transmitted initial-analysis result R1 is acquired by the acquisition function 111 of the image analysis support apparatus 1.

(Step S105) Subsequently, the image analysis support apparatus 1 determines a candidate of the additional-analysis application A2. Specifically, by implementing the determination function 112, the image analysis support apparatus 1 determines a candidate of the additional-analysis application A2 based on the initial-analysis result R1 obtained in step S104.

Let us assume, for example, a case where the initial-analysis application A1 analyzes image data relating to the head of a patient. In this case, the initial-analysis result R1 includes “brain tumor” as the lesion region extracted from the image data, and includes “analysis application P” as the name of the initial-analysis application A1 used for the extraction. The image analysis support apparatus 1 then determines an analysis application for analyzing “brain tumor” as a candidate of the additional-analysis application A2.

In addition, the image analysis support apparatus 1 estimates that the image data may include a lesion region different from the “brain tumor” based on the positional information of the “brain tumor” extracted by the initial-analysis application A1. Thus, the image analysis support apparatus 1 further determines an analysis application for analyzing “brain aneurysm” as a candidate of the additional-analysis application A2.

(Step S106) Subsequently, the image analysis support apparatus 1 transmits a candidate list. Specifically, by implementing the transmission function 115, the image analysis support apparatus 1 transmits display data including a list of the candidates relating to the additional-analysis application A2 and determined in step S105 to the operation terminal 2. The transmitted display data is acquired by the acquisition function 211 of the operation terminal 2.

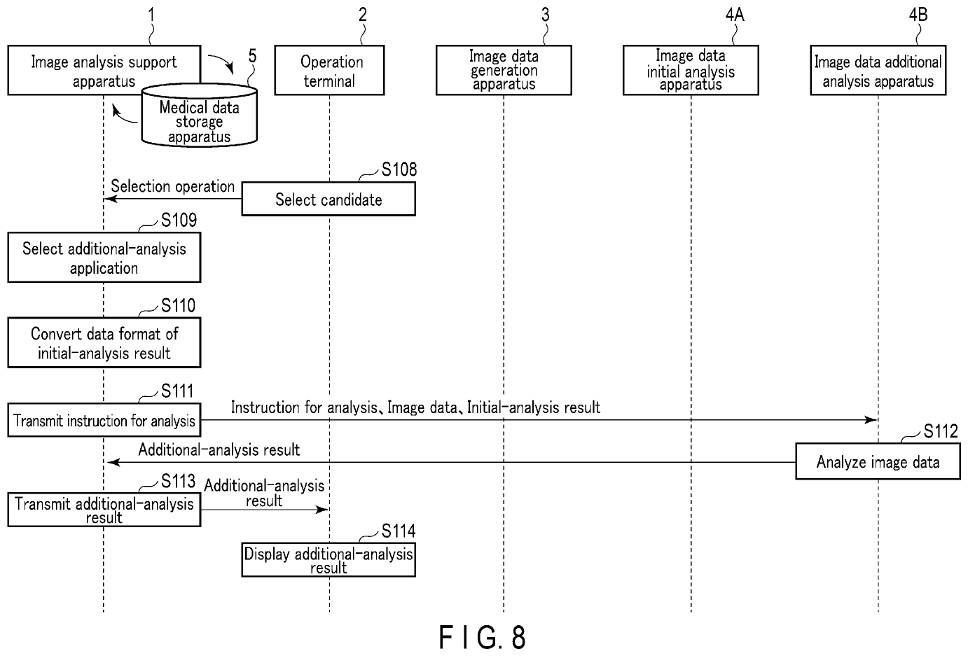

(Step S107) Subsequently, the operation terminal 2 displays a candidate list. Specifically, by implementing the display control function 212, the operation terminal 2 causes a display image based on the display data transmitted in step S106 to be displayed on the display 24. After step S107, the process proceeds to step S108 (see FIG. 8).

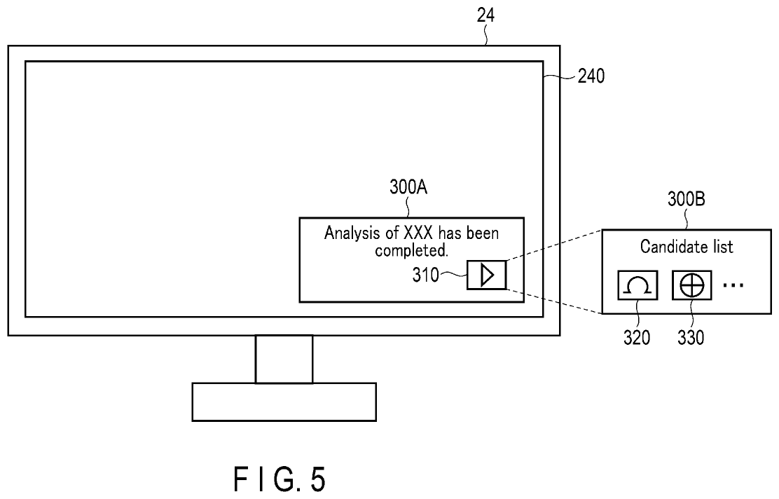

FIG. 5 is a diagram showing a first display example of a candidate list according to an embodiment. In this display example, a display image 300A is displayed as a pop-up on a display screen 240 of the display 24 of the operation terminal 2. The display image 300A includes a text indicating that the analysis of image data “XXX” has been completed and a button 310.

The operator may select the button 310 using the input IF 23. If the button 310 is selected, a display image 300B is displayed on the display screen 240. The display image 300B includes icons 320 and 330 as a candidate list of the additional-analysis application A2. Each of the icons 320 and 330 is given a mark unique to it.

The operator may select the icon 320 or 330 using the input IF 23. If the icon 320 is selected, the additional-analysis application A2 corresponding to the icon 320 is selected. If the icon 330 is selected, the additional-analysis application A2 corresponding to the icon 330 is selected.

According to this display example, multiple candidates relating to the additional-analysis application A2 are displayed through multiple icons. Each of the icons is given a mark unique to it. Thus, the operator can select a desired additional-analysis application A2 by visually distinguishing it from other additional-analysis applications A2. That is, the operator can efficiently select an additional-analysis application A2 that the operator wishes to use for additional analysis.

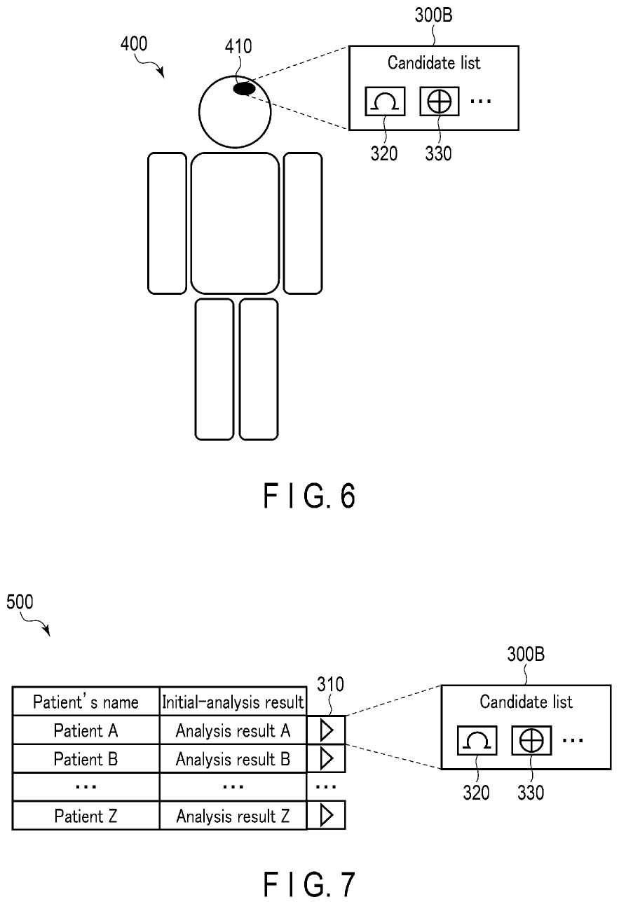

FIG. 6 is a diagram showing a second display example of a candidate list according to an embodiment. In this display example, a human body model 400 is displayed on the display screen 240. The human body model 400 is a three-dimensional human body model defined in a virtual space. The human body model 400 is rotated, magnified, or contracted in the virtual space according to the operation performed by the operator. The human body model 400 consists of body parts (head, torso, both arms, and both legs). Various anatomical structures as well as positional information are defined for the body parts.

The initial-analysis result R1 provided by the initial-analysis application A1 is superimposed on the human body model 400. For example, if the initial-analysis result R1 includes the positional information of “brain tumor”, a region 410 is superimposed on the position in the human body model 400 that corresponds to this positional information. The region 410 is superimposed on the brain region of the head of the human body model 400. Preferably, the region 410 is displayed in such a manner as to be visually distinguishable from the human body model 400 (e.g., color display, blink display).

The operator may select the region 410 using the input IF 23. If the region 410 is selected, the display image 300B is displayed on the display screen 240. As described above, the operator may select the icon 320 or 330 using the input IF 23.

In this display example, the initial-analysis result R1 is superimposed on the human body model 400. Thus, the operator can efficiently select an additional-analysis application A2 that the operator wishes to use for additional analysis while referring to the initial-analysis result R1.

FIG. 7 is a diagram showing a third display example of a candidate list according to an embodiment. In this display example, a table 500 is displayed on the display screen 240. The table 500 records, for each patient, the initial-analysis result R1 of image data provided by the initial-analysis application A1. The table 500 includes a column of “patient's name” and a column of “initial-analysis result”. Both of the columns include multiple records. In each of the records, a button 310 is arranged adjacently to the column of “initial-analysis result”.

The operator may select a desired button 310 using the input IF 23. As described above, if the button 310 is selected, the display image 300B is displayed on the display screen 240. The operator may select the icon 320 or 330 using the input IF 23. Instead of the icons, the names of the additional-analysis applications A2 may be displayed in a list on the display image 300B.

In this display example, the initial-analysis result R1 for each patient is displayed on the table 500. The buttons 310 for allowing the operator to select an additional-analysis application A2 are arranged adjacently to the table 500. Thus, the operator can efficiently select, for each of the patient's initial-analysis results R1, an additional-analysis application A2 that the operator wishes to use for additional analysis.

FIG. 8 is a sequence diagram showing a second operation example of the image analysis support system 100 according to the embodiment. FIG. 8 shows each step following step S107 shown in FIG. 4.

(Step S108) Subsequently, the operation terminal 2 selects a candidate. Specifically, by implementing the acquisition function 211, the operation terminal 2 acquires a selection operation performed on the candidate list of the additional-analysis application A2 displayed in step S107. The selection operation is input through the input IF 23. The operation terminal 2 transmits the input selection operation to the image analysis support apparatus 1. The transmitted selection operation is acquired by the acquisition function 111 of the image analysis support apparatus 1.

(Step S109) Subsequently, the image analysis support apparatus 1 selects the additional-analysis application A2. Specifically, by implementing the selection function 113, the image analysis support apparatus 1 selects the additional-analysis application A2 corresponding to the candidate selected in step S108. Thus, one additional-analysis application A2 is selected among the multiple additional-analysis applications A2 included in the image data additional analysis apparatus 4B.

(Step S110) Subsequently, the image analysis support apparatus 1 converts the data format of the initial-analysis result R1. Specifically, by implementing the conversion function 114, the image analysis support apparatus 1 converts the data format of the initial-analysis result R1 obtained in step 5104 such that the data format of the initial-analysis result R1 matches the data format that can be understood by the additional-analysis application A2 selected in step S109. Preferably, the image analysis support apparatus 1 converts the data format of the initial-analysis result R1 so that both of the data formats will be the same.

(Step S111) Subsequently, the image analysis support apparatus 1 transmits an instruction for analysis. Specifically, by implementing the transmission function 115, the image analysis support apparatus 1 transmits an instruction for analyzing the initial-analysis result R1 converted in step S110 to the image data additional analysis apparatus 4B. Simultaneously, by implementing the transmission function 115, the image analysis support apparatus 1 transmits the image data generated in step S102 and the initial-analysis result R1 converted in step S110 to the image data additional analysis apparatus 4B.

(Step S112) Subsequently, the image data additional analysis apparatus 4B analyzes the image data. Specifically, the image data additional analysis apparatus 4B additionally analyzes the image data transmitted in step S111 according to the instruction for analysis transmitted in step S111. The image data additional analysis apparatus 4B analyzes the image data using the initial-analysis result R1 transmitted in step S111 after starting the additional-analysis application A2 selected in step S109. The image data additional analysis apparatus 4B transmits, to the medical data storage apparatus 5, an additional-analysis result R2 of the image data provided by the additional-analysis application A2. The transmitted additional-analysis result R2 is acquired by the acquisition function 111 of the image analysis support apparatus 1.

(Step S113) Subsequently, the image analysis support apparatus 1 transmits the additional-analysis result R2. Specifically, by implementing the transmission function 115, the image analysis support apparatus 1 transmits the additional-analysis result R2 obtained in step S112 to the operation terminal 2. The transmitted additional-analysis result R2 is acquired by the acquisition function 211 of the operation terminal 2.

(Step S114) Lastly, the operation terminal 2 causes the additional-analysis result R2 to be displayed. Specifically, by implementing the display control function 212, the operation terminal 2 causes a display image based on the additional-analysis result R2 transmitted in step S113 to be displayed on the display 24. After step S114, the image analysis support system 100 ends the series of operations.

Above are descriptions of the image analysis support apparatus 1 and the image analysis support system 100 according to the embodiment. According to the embodiment, the image analysis support apparatus 1 determines a candidate relating to the additional-analysis application A2 based on the initial-analysis result R1 provided by the initial-analysis application A1. The image analysis support apparatus 1 causes the determined candidate to be displayed on the display.

Therefore, the image analysis support apparatus 1 can support an operator when the operator analyzes image data by combining multiple analysis applications. In particular, the image analysis support apparatus 1 determines a candidate of the additional-analysis application A2 based on the analysis result of the image data provided by the initial-analysis application A1 instead of the scan information of the image data. That is, the image analysis support apparatus 1 determines a candidate of the additional-analysis application A2 based on the actual analysis result. Thus, the operator can select a desired additional-analysis application A2.

Further, the image analysis support apparatus 1 converts the data format of the initial-analysis result R1 such that the data format of the initial-analysis result R1 matches the data format that can be understood by the additional-analysis application A2 selected by the operator. The image analysis support apparatus 1 transmits the converted initial-analysis result R1 to the additional-analysis application A2 selected by the operator.

Thus, the image analysis support apparatus 1 can make the additional-analysis application A2 that further analyzes the image data take over the analysis result of the image data provided by the initial-analysis application A1. This makes it unnecessary for the additional-analysis application A2 to perform, again, the same analysis as that performed by the initial-analysis application A1. That is, the image analysis support apparatus 1 can reduce the time for image data analysis since it eliminates the time for re-analysis of image data. Further, the additional-analysis application A2 can compute a new analysis result such that the new analysis result matches the analysis result provided by the initial-analysis application A1. That is, the image analysis support apparatus 1 can ensure the consistency between the two analysis results.

According to at least one embodiment described above, an image analysis using multiple analysis applications can be supported.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image analysis support apparatus comprising processing circuitry configured to:
acquire a first analysis result obtained through analysis of medical image data performed by a first analysis application; and
determine at least one candidate relating to a second analysis application based on the acquired first analysis result, the second analysis application further analyzing the medical image data.

2. The image analysis support apparatus according to claim 1, wherein the first analysis result includes at least one of: a name and a type of the first analysis application; or positions of an organ region, a lesion region and an anatomical landmark in the medical image data.

3. The image analysis support apparatus according to claim 1, wherein the processing circuitry selects, among the at least one candidate determined, one candidate according to a selection operation performed by an operator, and converts a data format of the first analysis result such that the data format of the first analysis result matches a data format that can be understood by the second analysis application corresponding to the selected candidate.

4. The image analysis support apparatus according to claim 3, wherein the processing circuitry transmits the first analysis result after the conversion to the second analysis application corresponding to the selected candidate.

5. An image analysis support system comprising an image analysis support apparatus and an operation terminal, wherein
the image analysis support apparatus comprises processing circuitry configured to:
acquire a first analysis result obtained through analysis of medical image data performed by a first analysis application; and
determine at least one candidate relating to a second analysis application based on the acquired first analysis result, the second analysis application further analyzing the medical image data, and
the operation terminal causes the at least one candidate determined by the image analysis support apparatus to be displayed.

6. An image analysis support method comprising:
acquiring a first analysis result obtained through analysis of medical image data performed by a first analysis application; and
determining at least one candidate relating to a second analysis application based on the acquired first analysis result, the second analysis application further analyzing the medical image data.
